(19) United States
(12) Patent Application Publication
(10) Pub. No.: US 2020/0376043 A1
(43) Pub. Date: Dec. 3, 2020

(54) Umbilical Cord Artery Perivascular Stem Cell Injection Solution for Treating Ischemic Diseases and Preparation Method Thereof

(71) Applicant: Nanjing Drum Tower Hospital, Nanjing (CN)

(72) Inventors: Lijun DING, Nanjing (CN); Haixiang SUN, Nanjing (CN); Lu XU, Nanjing (CN); Yifan LI, Nanjing (CN)

(73) Assignee: Nanjing Drum Tower Hospital, Nanjing (CN)

(21) Appl. No.: 16/770,073

(22) PCT Filed: Oct. 24, 2019
(86) PCT No.: PCT/CN2019/113020

§ 371 Date: Jun. 5, 2020

(30) Foreign Application Priority Data
May 5, 2019 (CN) 201910366815.4

(51) Int. Cl. A61K 35/51; A61P 9/10; A61K 35/16; A61K 35/19; A61K 9/00

(52) U.S. Cl. CPC A61K 35/51; A61P 9/10; A61K 9/0019; A61K 35/19; A61K 35/16

(57) ABSTRACT

An umbilical cord artery perivascular stem cell injection solution for treating ischemic diseases including suspended umbilical cord artery perivascular stem cells and a solvent and a preparation method thereof are provided. The solvent may be umbilical cord blood-derived platelet-rich plasma or injectable PBS containing human serum albumin. The preparation method includes: cultivating an umbilical cord artery tissue to derive perivascular stem cells, performing an enzymatic digestion to obtain individual umbilical cord artery perivascular stem cells, followed by washing twice with PBS, and gently mixing evenly with the solvent to obtain the umbilical cord artery perivascular stem cell injection solution. The cells are taken from the umbilical cord of medical waste and have the characteristics of easy separation, fast proliferation and low immunogenicity.

### CROSS REFERENCE TO THE RELATED APPLICATIONS

This application is the national phase entry of International Application No. PCT/CN2019/113020, filed on Oct. 24, 2019, which is based upon and claims priority to Chinese Patent Application No. 201910366815.4, filed on May 5, 2019, the entire contents of which are incorporated herein by reference.

### TECHNICAL FIELD

The present invention belongs to the field of medicine, relates to an injection solution, and more specifically to an umbilical cord artery perivascular stem cell injection solution for treating ischemic diseases and a preparation method thereof.

### BACKGROUND

Lower extremity ischemia is a common peripheral artery disease, which refers to a type of disease caused by a narrowing, a blockage, or an insufficient blood perfusion in lower extremity arteries due to various reasons, resulting in intermittent claudication, ulcers, gangrene and other ischemic manifestations of the lower extremities. At present, there is no effective treatment and control method for treating ischemic diseases of the lower extremities, which is insufficient to induce enough neovascularization in a wide range of ischemic and hypoxic regions. Finding high-quality stem cells for transplantation to improve the blood perfusion in ischemic lower extremities has become an important issue to be solved urgently.

Currently, the research of human umbilical cord mesenchymal stem cells still has the following major drawbacks. (1) The umbilical cord Wharton's jelly mesenchymal stem cell or cord stem cell mixture mainly used in current clinical use has unclear characteristics, low CD146 expression on the cell surface, and weak angiogenesis. (2) Rehabilitation of patients with lower extremity ischemia depends on the reconstruction of adequate blood supply, and the application of human umbilical cord Wharton's jelly mesenchymal stem cells in lower extremity ischemia is not effective.

### SUMMARY

The present invention provides an umbilical cord artery perivascular stem cell injection solution for treating ischemic diseases and a preparation method thereof to overcome the drawbacks of the prior art.

To achieve the above objective, the present invention provides an umbilical cord artery perivascular stem cell injection solution for treating ischemic diseases, including suspended umbilical cord artery perivascular stem cells and a solvent.

Further, the umbilical cord artery perivascular stem cell injection solution for treating ischemic diseases of the present invention may further have the following characteristic: the solvent is umbilical cord blood-derived platelet-rich plasma.

Further, the umbilical cord artery perivascular stem cell injection solution for treating ischemic diseases of the present invention may further have the following characteristic: a content of the umbilical cord artery perivascular stem cells is 5×10⁶/mL, wherein 5×10⁶/mL refers to 5×10⁶ umbilical cord artery perivascular stem cells suspended in 1 mL of solvent.

Further, the umbilical cord artery perivascular stem cell injection solution for treating ischemic diseases of the present invention may further have the following characteristic: the solvent is injectable phosphate-buffered saline (PBS) containing human serum albumin.

Further, the umbilical cord artery perivascular stem cell injection solution for treating ischemic diseases of the present invention may further have the following characteristic: a content of the umbilical cord artery perivascular stem cells is 5×10⁶/mL, and a content of the human serum albumin is 5% (w/v) of the volume of PBS.

The present invention further provides a method for preparing the above-mentioned umbilical cord artery perivascular stem cell injection solution for treating ischemic diseases, including: cultivating an umbilical cord artery tissue to derive perivascular stem cells, then performing an enzymatic digestion to obtain individual umbilical cord artery perivascular stem cells, followed by washing twice with PBS, and gently mixing with the umbilical cord blood-derived platelet-rich plasma evenly to obtain the umbilical cord artery perivascular stem cell injection solution.

Further, in the present invention, the method for preparing the above-mentioned umbilical cord artery perivascular stem cell injection solution for treating ischemic diseases may further have the following characteristic: the preparation process of the umbilical cord artery perivascular stem cells includes: collecting 7-15 cm of a fresh human umbilical cord after a delivery of a full-term newborn, and then transporting to a laboratory with PBS containing 10% (w/v) penicillin-streptomycin and 1% (v/v) heparin at 4° C. on ice within 4 h; then, taking out the umbilical cord in an ultra-clean workbench environment, squeezing out the blood from the umbilical cord, and washing with PBS repeatedly until the umbilical cord is free of blood and blood clots, then trimming two sections of the umbilical cord, cutting the umbilical cord along a long axis direction of the blood vessel in the umbilical cord, and separating the umbilical artery by blunt dissection with forceps; subsequently, cutting the umbilical artery in a direction perpendicular to the umbilical artery to tissue blocks of 1-2 mm³ in size, spreading the tissue blocks evenly on a bottom of a 100 mm cell culture dish, and then placing the 100 mm cell culture dish in an incubator with 5% CO₂ and saturated humidity at 37° C. for 3 h; then, slowly adding 5 mL of Dulbecco's Modified Eagle Medium-low glucose (DMEM-LG) to the 100 mm cell culture dish and gently placing the 100 mm cell culture dish in the incubator for cultivation; adding 3 mL DMEM-LG medium on the 3^{rd} day of the first week of cultivation and adding 2 mL of DMEM-LG medium on the 7^{th} day of the first week of cultivation; after that, exchanging half-volume of the fluid in the 100 mm cell culture dish with fresh DMEM-LG medium twice in the second week of cultivation, and then exchanging the whole-volume of the fluid twice in the third week of cultivation, and during the third week of cultivation, observing the spreading of adherent cells from the edge of the tissue blocks; after 3 weeks of cultivation, when the cells grow to 80% confluence, removing the umbilical artery tissue blocks, then digesting with 0.05% (w/v) trypsin for subculture; and harvesting P3-P5 generation cells for preparing injection suspensions.

Further, in the present invention, the method for preparing the above-mentioned umbilical cord artery perivascular stem cell injection solution for treating ischemic diseases may further have the following characteristic: a method for preparing the umbilical cord blood-derived platelet-rich plasma includes: adding 750 international units (IU) of heparin sodium to a 50 mL centrifuge tube to collect 40 mL of umbilical cord blood; performing a centrifugation at a low speed of 200 g for 10-15 min at room temperature, then collecting an upper portion of the resultant liquid; centrifuging the upper liquid portion at a high speed of 2000 g for 10 min again to cause platelets to deposit at a bottom of the centrifuge tube; then, discarding part of supernatant plasma, retaining 200 μL of the plasma and platelets deposited at the bottom of the tube, and gently resuspending to obtain the umbilical cord blood-derived platelet-rich plasma (PRP).

Further, in the present invention, the method for preparing the above-mentioned umbilical cord artery perivascular stem cell injection solution for treating ischemic diseases may further have the following characteristic: wherein, washing the obtained individual umbilical cord artery perivascular stem cell suspension twice with PBS without calcium and magnesium, then resuspending the cell precipitate with the umbilical cord blood-derived platelet-rich plasma, and then gently mixing evenly to obtain the umbilical cord artery perivascular stem cell injection solution; wherein the umbilical cord artery perivascular stem cell injection solution is placed at 4° C. for short-term storage, used within 12 h, and shaken gently before use.

The present invention further provides a method for preparing the above-mentioned umbilical cord artery perivascular stem cell injection solution for treating ischemic diseases, including: cultivating an umbilical cord artery tissue to derive perivascular stem cells, then performing an enzymatic digestion to obtain individual umbilical cord artery perivascular stem cells, followed by washing twice with PBS without calcium and magnesium, and resuspending the cell precipitate with PBS containing 5% (w/v) human serum albumin, and then gently mixing evenly to obtain the umbilical cord artery perivascular stem cell injection solution; wherein, the umbilical cord artery perivascular stem cell injection solution is placed at 4° C. for short-term storage, used within 12 h, and is shaken gently before use.

The advantages of the present invention are as follows. The present invention provides an umbilical cord artery perivascular stem cell injection solution for treating ischemic diseases and a preparation method thereof. Without the aid of growth factors, the umbilical cord artery perivascular stem cells can form bud-like or tube-like structures similar to blood vessels or vasculature spontaneously in vivo, and can stimulate vascular endothelial cells to form vascular tube-like structures in vivo as well. In addition, there are numerous angiogenic factors in the umbilical cord blood-derived platelet-rich plasma (PRP), which are involved in angiogenesis. PRP also contains numerous cytokines and proteins that can promote the proliferation, differentiation, migration, and adhesion of stem cells. The umbilical cord artery perivascular stem cells and the umbilical cord blood-derived platelet-rich plasma have a remarkable synergistic effect. Lower extremity ischemia mouse models were subjected to a treatment by transplanting the human umbilical cord artery perivascular stem cell injection solution resuspended in the umbilical cord blood-derived platelet-rich plasma, and the results showed that the blood supply of the ischemic lower extremities of the mouse models was significantly improved.

### BRIEF DESCRIPTION OF THE DRAWINGS

FIG. 1A-1 to FIG. 1A-6 show immunofluorescence staining images of vWF, mCD31, and hCD31 in Matrigel plugs of human umbilical cord artery perivascular stem cell group and Wharton's jelly mesenchymal stem cell group;

FIG. 1B-1 to FIG. 1B-3 are diagrams showing quantitative statistics of immunofluorescence staining of vWF, mCD31, and hCD31 in Matrigel plugs of human umbilical cord artery perivascular stem cell group and Wharton's jelly mesenchymal stem cell group;

FIG. 1C-1 and FIG. 1C-2 show immunohistochemical staining images of mCD31 in Matrigel plugs of human umbilical cord artery perivascular stem cell group and Wharton's jelly mesenchymal stem cell group;

FIG. 1D is a diagram showing quantitative statistics of immunofluorescence staining of mCD31 in Matrigel plugs of human umbilical cord artery perivascular stem cell group and Wharton's jelly mesenchymal stem cell group;

FIG. 2A-1 to FIG. 2A-3 show immunohistochemical staining images of mCD31 of ischemic gastrocnemius muscle in transplanted groups of human umbilical cord Wharton's jelly mesenchymal stem cell injection solution, human umbilical cord artery perivascular stem cell injection solution, and PBS;

FIG. 2B-1 and FIG. 2B-2 are diagrams showing quantitative statistics of immunofluorescence staining of mCD31 of ischemic gastrocnemius muscle in transplanted groups of human umbilical cord Wharton's jelly mesenchymal stem cell injection solution, human umbilical cord artery perivascular stem cell injection solution, and PBS;

FIG. 3A-1 and FIG. 3A-2 show Doppler ultrasound blood flow images of ischemic lower extremities of mice transplanted with human umbilical cord Wharton's jelly mesenchymal stem cell injection solution and human umbilical cord artery perivascular stem cell injection solution;

and

FIG. 3B is a diagram showing blood flow quantification results of ischemic lower extremities of mice transplanted with human umbilical cord Wharton's jelly mesenchymal stem cell injection solution and human umbilical cord artery perivascular stem cell injection solution.

### DETAILED DESCRIPTION OF THE EMBODIMENTS

The present invention is further described below with reference to the specific embodiments.

### Embodiment 1

An umbilical cord artery perivascular stem cell injection solution for treating ischemic diseases includes suspended umbilical cord artery perivascular stem cells and umbilical cord blood-derived platelet-rich plasma, wherein a content of the umbilical cord artery perivascular stem cells is 5×10⁶/mL.

A method for preparing the umbilical cord artery perivascular stem cell injection solution for treating ischemic diseases includes: an umbilical cord artery tissue is cultivated to derive perivascular stem cells, an enzymatic digestion is carried out to obtain individual umbilical cord artery perivascular stem cells, followed by washing twice with PBS without calcium and magnesium, resuspending the cell precipitate with the umbilical cord blood-derived platelet-rich plasma, and gently mixing evenly, to obtain the umbilical cord artery perivascular stem cell injection solution. The umbilical cord artery perivascular stem cell injection solution is placed at 4° C. for short-term storage, used within 12 h, and shaken gently before use.

A method for preparing the umbilical cord artery perivascular stem cells includes: after a delivery of a full-term newborn, 7-15 cm of a fresh human umbilical cord is collected, and then transported to a laboratory with PBS containing 10% (w/v) penicillin-streptomycin and 1% (v/v) heparin at 4° C. on ice within 4 h. Then, the umbilical cord is taken out in an ultra-clean workbench environment, the blood in the umbilical cord is squeezed out, and the umbilical cord is washed with PBS repeatedly until the umbilical cord is free of blood and blood clots. Subsequently, two sections of the umbilical cord are trimmed, the umbilical cord is cut along a long axis direction of the blood vessel in the umbilical cord, and the umbilical artery is separated by blunt dissection with forceps. Next, the umbilical artery is cut in a direction perpendicular to the umbilical artery to tissue blocks of 1-2 mm³ in size. The tissue blocks are spread evenly on a bottom of a 100 mm cell culture dish, and the 100 mm cell culture dish is placed in an incubator with 5% CO₂ and saturated in humidity at 37° C. for 3 h. Then, 5 mL of DMEM-LG medium is slowly added to the 100 mm cell culture dish, and the 100 mm cell culture dish is gently placed in the incubator for cultivation. 3 mL of DMEM-LG medium is added on the 3^{rd} day of the first week of cultivation and 2 mL of DMEM-LG medium is added on the 7^{th} day of the first week of cultivation. After that, the half-volume of the fluid in the 100 mm cell culture dish is exchanged with fresh DMEM-LG medium twice in the second week of cultivation, and the whole-volume of the fluid is exchanged twice in the third week of cultivation. During the third week of cultivation, the spreading of adherent cells from the edge of the tissue blocks is observed. After 3 weeks of cultivation, when the cells grow to 80% confluence, the umbilical artery tissue blocks are removed, then the cells are digested with 0.05% (w/v) trypsin for subculture. P3-P5 generation cells are harvested for preparing injection suspensions.

A method for preparing the umbilical cord blood-derived platelet-rich plasma includes: 750 IU of heparin sodium is added to a 50 mL centrifuge tube to collect 40 mL of umbilical cord blood. A centrifugation is performed at a low speed of 200 g for 10-15 min at room temperature, and then an upper liquid is collected. Subsequently, the upper liquid is centrifuged at a high speed of 2000 g for 10 min again to cause platelets to deposit at a bottom of the centrifuge tube. After that, part of supernatant plasma is discarded, and 200 μL of the plasma and platelets deposited at the bottom is retained and then gently resuspended to obtain the umbilical cord blood-derived platelet-rich plasma (PRP).

A method for applying the umbilical cord artery perivascular stem cell injection solution for treating ischemic diseases includes: applying an injection. Specifically, the umbilical cord artery perivascular stem cell injection solution is used for a multi-site injection solution into the gastrocnemius muscle.

In vivo Matrigel plug assay on umbilical cord artery perivascular stem cells:

1. Randomly Grouping of BALB/C Immunodeficient Mice

A total of 20 mice in various litters were sorted according to their initial body weights. 20 two-digit random numbers were randomly generated by a computer, and the numbers were sorted correspondingly to the body weights. Five groups were randomly assigned with 4 mice in each group. After grouping, there was no statistical difference in the average weight of each group. According to the requirements of stocking density, each group including 4 mice was fed in a cage separately. The group number and cage number were marked, and the serial number of the mice within each cage was marked with picric acid. After grouping, the mice were fed for one week to adapt to the environment, and the living conditions and weight changes of the mice were observed. After one week, the mice in each group had satisfactory weight gain, healthy and active appearance, and gentleness without irritability. The average body weight was 20-30 g. After reaching 7-9 weeks of age, there was no significant difference in the average body weight of each group. These mice met the criteria for entering the experiment and were used in the following experiment.

2. Establishment of In Vivo Angiogenesis Models

After anesthesia, the mice were injected with cell suspensions subcutaneously under the abdomen along the groin area, and divided into 4 groups, including: (1) an umbilical cord artery perivascular stem cell group: 200 μL of Matrigel matrix+50 μL of PBS containing 7.5×10⁵ umbilical cord artery perivascular stem cells (UCA-PSCs); (2) an umbilical cord Wharton's jelly mesenchymal stem cell group: 200 μL of Matrigel matrix+50 μL of PBS containing 7.5×10⁵ umbilical cord Wharton's jelly mesenchymal stem cells (WJ-MSCs); (3) a negative control group: 200 μL of Matrigel matrix+50 μL of PBS; and (4) a positive control group: 200 μL of Matrigel matrix+50 μL of PBS containing 37.5 ng of basic fibroblast growth factor (bFGF).

3. Immunofluorescence Staining on Frozen Sections

After 14 days, the mice were sacrificed, and the subcutaneous Matrigel plugs of each group were taken out. The tissue block was placed flat in a small soft plastic box (about 2 cm in diameter). An Opti-mum Cutting Temperature (OCT) embedding agent may be added to immerse the tissue in an appropriate amount thereof, and then the special box was slowly placed into a small cup containing liquid nitrogen. When the bottom of the box is in contact with the liquid nitrogen, the small box was kept in place and prevented from entering the liquid nitrogen. After being kept for about 10-20 seconds, the tissue was frozen into a frozen block. After the frozen block is made, it was placed in a constant freezing slicer for frozen sectioning. The knife chamber of the constant freezing slicer (Leica) was set to −25° C., and the machine chamber was set to −20° C. The anti-roll plate was placed in an appropriate position and angle. The slides were attached to the tissue sections, and prevented from moving up or down.

After being placed at room temperature for 30 min, the sections were fixed with acetone at 4° C. for 5-10 min. Then, the sections were rinsed with PBS for 3 times, with 5 min for each time. The frozen sections were air-dried at room temperature for 15 min, and were blocked with PBS containing 10% (v/v) normal goat serum for 1 h at room temperature. Then, an appropriately diluted primary antibody or primary antibody working solution (mCD31, Abcam; hCD31, BD; and vWF, Abcam) was added dropwise to evenly cover the tissue surface, preventing the tissue from drying up throughout the process. Then, the sections were placed in an immunohistochemical wet box added with PBS and incubated for 2 h at room temperature or overnight at 4° C. On the next day, the wet box was first placed at 37° C. for 1 h, and then the primary antibody on the sections was suctioned for recovery, and the sections were inserted into a small staining tank and then rinsed with PBS. After that, the secondary antibody, Alexa Fluor 488-conjugated goat anti-rabbit IgG (1:200, Invitrogen)/Alexa Fluor 555-conjugated goat anti-rabbit IgG (1:200, Invitrogen), diluted with PBS was added dropwise onto the sections, and then the sections were placed in an oven and incubated at 37° C. for 1 h (keeping away from light). Then, the sections were placed in the staining tank and then rinsed with PBS for 3 times, with 5 min for each time. A 4′,6-diamidino-2-phenylindole (DAPI) working solution was added onto the sections to stain the nucleus at room temperature for 10-20 min (staining at a working concentration of 0.1% for 15 min). Then, the DAPI was recovered, 5-10 μL of an anti-fluorescence fading agent or neutral gum was added dropwise onto the slides, and the sections were mounted with clean coverslips, and then the tissue sections were observed and photographed under a fluorescence microscope or a confocal microscope. The mounted sections were placed in a section box and placed in a 4° C. refrigerator, and stored for about a week.

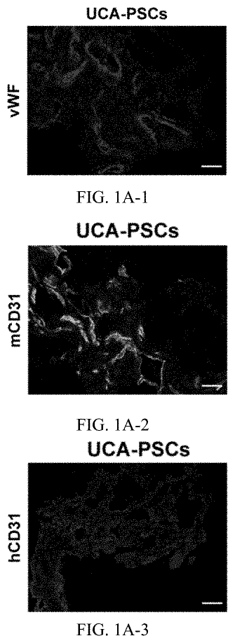

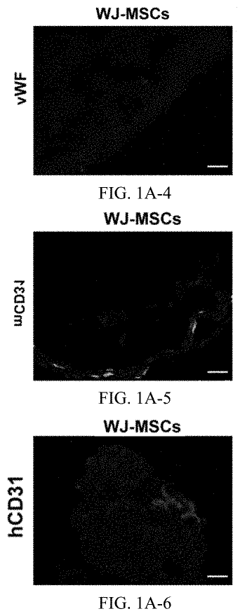

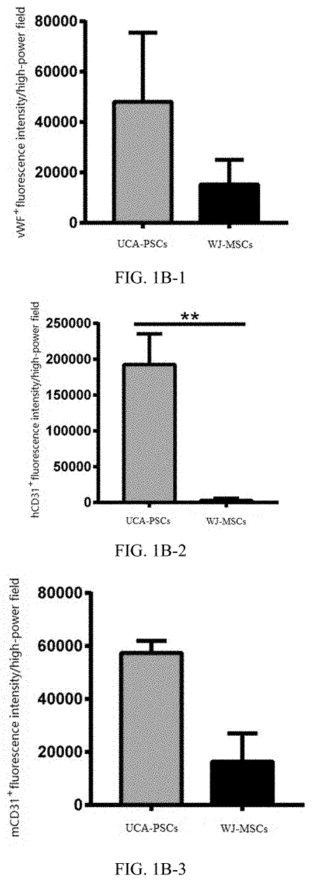

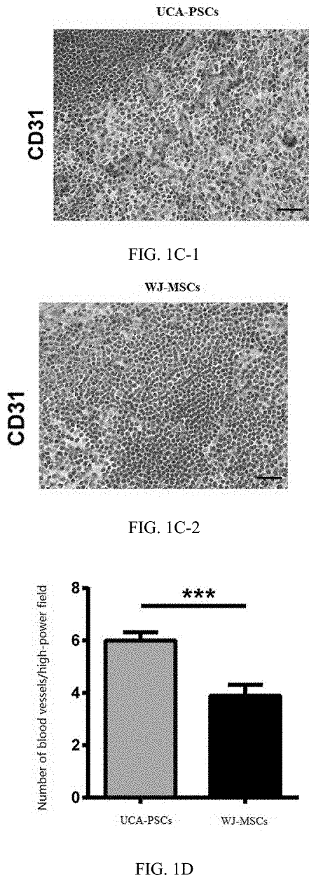

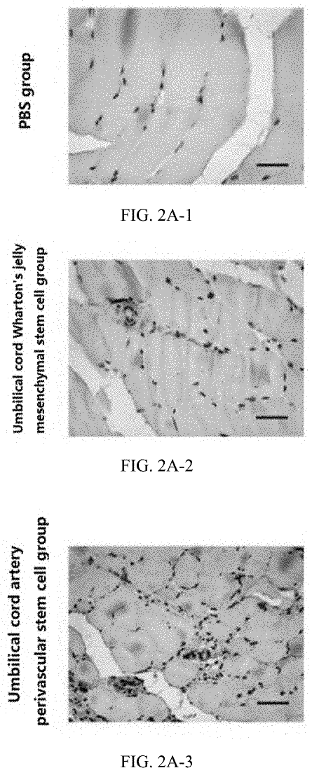

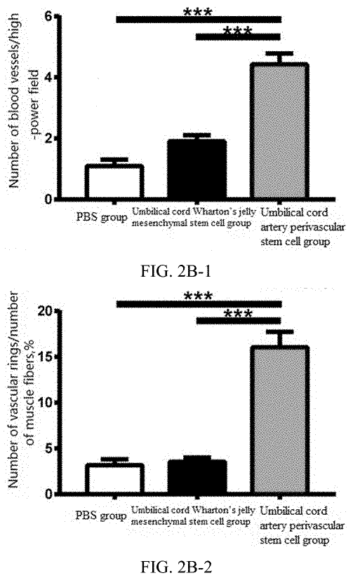

FIG. 1A-1 to FIG. 1A-6 show immunofluorescence staining images of vWF, mCD31, and hCD31 in Matrigel plugs of the human umbilical cord artery perivascular stem cell group and the Wharton's jelly mesenchymal stem cell group; FIG. 1B-1 to FIG. 1B-3 are diagrams showing quantitative statistics of immunofluorescence staining of vWF, mCD31, and hCD31 in Matrigel plugs of the human umbilical cord artery perivascular stem cell group and the Wharton's jelly mesenchymal stem cell group; FIG. 1C-1 and FIG. 1C-2 show immunohistochemical staining images of mCD31 in Matrigel plugs of the human umbilical cord artery perivascular stem cell group and the Wharton's jelly mesenchymal stem cell group; and FIG. 1D is a diagram showing quantitative statistics of immunofluorescence staining of mCD31 in Matrigel plugs of the human umbilical cord artery perivascular stem cell group and the Wharton's jelly mesenchymal stem cell group;

For the comparison of the ability of the human umbilical cord artery perivascular stem cells and the umbilical cord Wharton's jelly mesenchymal stem cells to form a ring and induce ring formation in vivo, as shown in FIGS. 1a, 1b and 1c, the immunofluorescence results of vWF, mCD31, and hCD31 showed that after the human umbilical cord artery perivascular stem cells embedded in the Matrigel matrix were transplanted into the groin subcutaneous area of the immunodeficient mice, the number of new blood vessels in the gel block was more than that of the Wharton's jelly mesenchymal stem cells. As shown in FIG. 1D, the data quantification results showed that the fluorescence intensity per high-power field of the human umbilical cord artery perivascular stem cell group was also significantly higher than that of the human umbilical cord Wharton's jelly mesenchymal stem cell group. The results suggest that the abilities of the human umbilical cord artery perivascular stem cells to self-angiogenesis in vivo and to stimulate vascularization of mouse endothelial cells are significantly better than that of the Wharton's jelly mesenchymal stem cells.

Experimental study of the umbilical cord artery perivascular stem cell injection solution for the treatment of lower extremity ischemia mouse models:

1. Experimental Animals

Six- to eight-week-old male BALB/C nude mice were housed in an individually ventilated cage (IVC) animal room of the Experimental Animal Center of Drum Tower Hospital affiliated to Medical College of Nanjing University, at a room temperature of 22° C., and were kept under a 12-hour light/dark cycle (06: 00-18:00). All the mice were allowed free access to water and food.

2. Construction of Models

After weighing the mice with a weighing tray, the mice were anesthetized by intraperitoneal injection with 1% chloral hydrate at 0.3 mL/kg. After being anesthetized, the mice were placed in a supine position and fixed onto a surgical board with rubber bands, followed by conventional skin preparation, iodine sterilization, and laying sterile hole towels. Then, the skin was gently lifted with ophthalmic forceps, and a longitudinal incision of about 5 mm in length was made from the groin to the inner thigh along the direction of the blood vessel with ophthalmic scissors. Under 20× magnification of a dissecting microscope, the membranous vascular sheath was gently punctured by sharp forceps to expose the femoral artery, vein and their branches. Then, the distal end of the common femoral artery and its branches were ligatured with a suture of size 7.

3. Experimental Grouping

Four weeks after the nude mice were modeled, they were randomly divided into three groups, including: a PBS group, an umbilical cord Wharton's jelly mesenchymal stem cell-containing PRP group (with the umbilical cord Wharton's jelly mesenchymal stem cell injection solution), and an umbilical cord artery perivascular stem cell-containing PRP group (with the umbilical cord artery perivascular stem cell injection solution), while a sham-surgery group was used as a control. Five sizes of the gastrocnemius muscle of ischemic lower extremity were randomly selected. The PBS group was injected with 100 μL of PBS; the umbilical cord Wharton's jelly mesenchymal stem cell-containing PRP group was injected with 100 μL of a PRP suspension mixed with 5×10⁶ umbilical cord Wharton's jelly mesenchymal stem cells; and the umbilical cord artery perivascular stem cell-containing PRP group was injected with 100 μL of a PRP suspension mixed with 5×10⁶ umbilical cord artery perivascular stem cells. The nude mice were housed in the IVC animal room after the surgery.

4. Counting of Microvessel Density by Immunohistochemistry

The muscle tissue of the gastrocnemius muscle in the ischemic lower extremities of the nude mice was fixed with 4% paraformaldehyde. The ischemic lower extremities of the nude mice were sectioned continuously with a thickness of 5 In every five sections, one section was selected for hematoxylin-eosin (HE) staining, and another section was selected for immunohistochemical staining. Five high-power fields (100×) were randomly selected from each section to count the number of blood vessels stained brown by CD31 antibody. Any brown-stained vascular endothelial cell or cluster of vascular endothelial cells was defined as a blood vessel count. The vessels with a vascular diameter greater than eight red blood cells or a relatively thick muscle layer were not counted.

5. Detection of Blood Flow Changes by Laser Doppler Instrument

After being successfully anesthetized, the mice were placed in a prone position on a gray soft pad specially designed for the laser Doppler instrument, and their feet were fixed with a double-sided tape so that the soles of their feet were facing upward and symmetrical. The position of the scanning probe was adjusted directly above the mice to be about 15 cm away from the mice, and then the blood flow index was detected by the laser Doppler instrument.

FIG. 2A-1 to FIG. 2A-3 show immunohistochemical staining images of mCD31 of ischemic gastrocnemius muscle in transplanted groups of the human umbilical cord Wharton's jelly mesenchymal stem cell injection solution, the human umbilical cord artery perivascular stem cell injection solution, and PBS. FIG. 2B-1 and FIG. 2B-2 are diagrams showing quantitative statistics (including a number of vascular rings per high-power field, and a ratio of the number of the vascular rings per high-power field to a number of muscle fibers) of immunofluorescence staining of mCD31 of ischemic gastrocnemius muscle in transplanted groups of the human umbilical cord Wharton's jelly mesenchymal stem cell injection solution, the human umbilical cord artery perivascular stem cell injection solution, and PBS.

For the comparison of the number of new blood vessels in the gastrocnemius muscle of the mice after being transplanted with the human umbilical cord artery perivascular stem cell injection solution and the umbilical cord Wharton's jelly mesenchymal stem cell injection solution, as shown in FIG. 2A-1 to FIG. 2A-3, the immunohistochemical results of mCD31 showed that the number of new blood vessels of ischemic lower extremities in the human umbilical cord artery perivascular stem cell injection group was higher than that in the umbilical cord Wharton's jelly mesenchymal stem cell injection group and the PBS group. As shown in FIG. 2b, the data quantification results showed that the number of vascular rings per high-power field and the ratio of the number of vascular rings per high-power field to the number of muscle fibers in the human umbilical cord artery perivascular stem cell injection group were significantly higher than those in the umbilical cord Wharton's jelly mesenchymal stem cell injection group. The results suggest that the human umbilical cord artery perivascular stem cell PRP injection solution significantly improved the angiogenesis in ischemic lower extremities of mice.

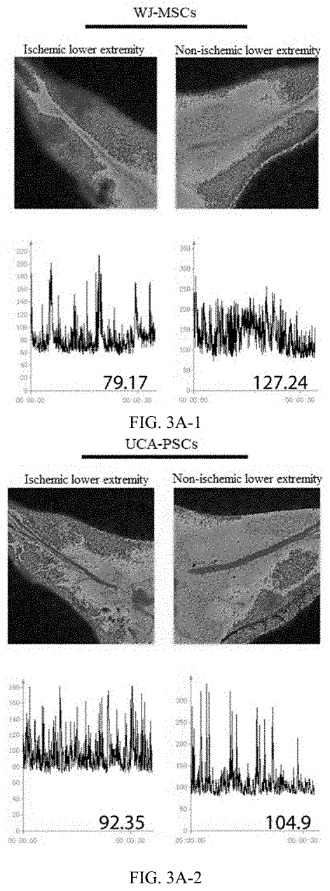

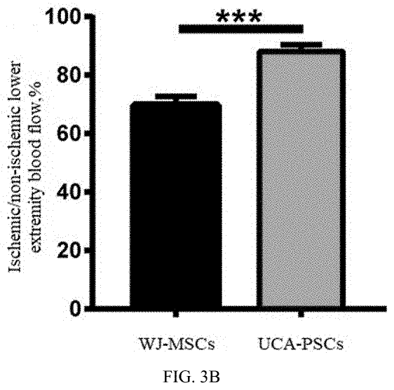

FIG. 3A-1 and FIG. 3A-2 show Doppler ultrasound blood flow images of ischemic lower extremities of mice transplanted with the human umbilical cord Wharton's jelly mesenchymal stem cell injection solution and the human umbilical cord artery perivascular stem cell injection solution. FIG. 3B is a diagram showing blood flow quantification results of ischemic lower extremities of mice transplanted with the human umbilical cord Wharton's jelly mesenchymal stem cell injection solution and the human umbilical cord artery perivascular stem cell injection solution.

For the comparison of blood flow of gastrocnemius muscle in mice after being transplanted with the human umbilical cord artery perivascular stem cell injection solution and the umbilical cord Wharton's jelly mesenchymal stem cell injection solution, as shown in FIGS. 3a and 3b, the Doppler ultrasound blood flow images and the data quantification results show that the blood flow abundance of the ischemic lower extremity in the human umbilical cord artery perivascular stem cell injection group was significantly higher than that in the umbilical cord Wharton's jelly mesenchymal stem cell injection group and the PBS group. The results suggest that the ability of the human umbilical cord artery perivascular stem cell injection solution to improve blood supply to ischemic lower extremities of mice is significantly better than that of the Wharton's jelly mesenchymal stem cell injection solution.

### Embodiment 2

An umbilical cord artery perivascular stem cell injection solution for treating ischemic diseases includes: suspended umbilical cord artery perivascular stem cells and injectable PBS containing human serum albumin, wherein a content of the umbilical cord artery perivascular stem cells is 5×10⁶/mL and a content of the human serum albumin is 5% (w/v) of the volume of PBS.

A method for preparing the umbilical cord artery perivascular stem cell injection solution for treating ischemic diseases includes: an umbilical cord artery tissue is cultivated to derive perivascular stem cells, an enzymatic digestion is carried out to obtain individual umbilical cord artery perivascular stem cells, followed by washing twice with PBS without calcium and magnesium, resuspending the cell precipitate with the PBS containing 5% (w/v) human serum albumin, and gently mixing evenly, to obtain the umbilical cord artery perivascular stem cell injection solution. The umbilical cord artery perivascular stem cell injection solution is placed at 4° C. for short-term storage, used within 12 h, and shaken gently before use.

What is claimed is:

1. An umbilical cord artery perivascular stem cell injection solution for treating ischemic diseases, comprising:
individual umbilical cord artery perivascular stem cells in a suspended state and a solvent.

2. The umbilical cord artery perivascular stem cell injection solution for treating the ischemic diseases according to claim 1, wherein:
the solvent is umbilical cord blood-derived platelet-rich plasma.

3. The umbilical cord artery perivascular stem cell injection solution for treating the ischemic diseases according to claim 2, wherein:
a content of the individual umbilical cord artery perivascular stem cells is 5×10⁶/mL.

4. The umbilical cord artery perivascular stem cell injection solution for treating the ischemic diseases according to claim 1, wherein:
the solvent is phosphate buffer saline (PBS) containing human serum albumin.

5. The umbilical cord artery perivascular stem cell injection solution for treating the ischemic diseases according to claim 4, wherein:
a content of the individual umbilical cord artery perivascular stem cells is 5×10⁶/mL; and
a content of the human serum albumin is 5 grams per 100 mL of the PBS.

6. A method for preparing the umbilical cord artery perivascular stem cell injection solution for treating the ischemic diseases according to claim 2, comprising:
cultivating an umbilical cord artery tissue to derive perivascular stem cells, then performing an enzymatic digestion on the perivascular stem cells to obtain the individual umbilical cord artery perivascular stem cells, followed by washing the individual umbilical cord artery perivascular stem cells twice with PBS, and gently mixing the individual umbilical cord artery perivascular stem cells with the umbilical cord blood-derived platelet-rich plasma evenly to obtain the umbilical cord artery perivascular stem cell injection solution.

7. The method according to claim 6, wherein:
a process for preparing the individual umbilical cord artery perivascular stem cells comprises:
collecting 7-15 cm of a fresh human umbilical cord after a delivery of a full-term newborn, and then transporting the fresh human umbilical cord to a laboratory with PBS containing 10% (w/v) penicillin-streptomycin and 1% (v/v) heparin at 4° C. on ice within 4 h;
taking out the fresh human umbilical cord in an ultra-clean workbench environment, squeezing blood out of the fresh human umbilical cord, and washing the fresh human umbilical cord with PBS repeatedly until the fresh human umbilical cord is free of blood and blood clots, then trimming two sections of the fresh human umbilical cord, cutting the fresh human umbilical cord along a long axis direction of a blood vessel in the fresh human umbilical cord, and separating an umbilical artery by a blunt dissection with forceps;
cutting the umbilical artery in a direction perpendicular to the umbilical artery to obtain tissue blocks of 1-2 mm³ in size, spreading the tissue blocks evenly on a bottom of a 100 mm cell culture dish, and then placing the 100 mm cell culture dish in an incubator with 5% CO₂ and saturated humidity at 37° C. for 3 h;
then, slowly adding 5 mL of DMEM-LG medium to the 100 mm cell culture dish and gently placing the 100 mm cell culture dish in the incubator for a cultivation;
adding 3 mL of DMEM-LG medium to the 100 mm cell culture dish on the 3^{rd} day of a first week of the cultivation and then adding 2 mL of DMEM-LG medium to the 100 mm cell culture dish on the 7^{th} day of the first week of the cultivation; then, exchanging a half-volume of fluid in the 100 mm cell culture dish with fresh DMEM-LG medium twice in a second week of the cultivation, and then exchanging a whole-volume of the fluid twice in a third week of the cultivation, and during the third week of the cultivation, observing a spreading of adherent cells from an edge of the tissue blocks;
after 3 weeks of the cultivation, when the adherent cells grow to 80% confluence, removing the tissue blocks, then digesting the adherent cells with 0.05% (w/v) trypsin for a subculture; and
harvesting P3-P5 generation cells of the adherent cells for the umbilical cord artery perivascular stem cell injection solution.

8. The method according to claim 6, wherein:
a process for preparing the umbilical cord blood-derived platelet-rich plasma comprises:
adding 750 IU of heparin sodium to a 50 mL centrifuge tube to collect 40 mL of umbilical cord blood;
performing a centrifugation on the umbilical cord blood at a speed of 200 g for 10-15 min at room temperature to obtain a resultant liquid, then collecting an upper portion of the resultant liquid;
centrifuging the upper portion of the resultant liquid at a speed of 2000 g for 10 min to cause platelets in the upper portion of the resultant liquid to deposit at a bottom of the 50 mL centrifuge tube to obtain supernatant plasma; then, discarding part of the supernatant plasma, retaining a total 200 μL of the supernatant plasma and the platelets deposited at the bottom, and gently resuspending the total 200 μL of the supernatant plasma and the platelets to obtain the umbilical cord blood-derived platelet-rich plasma.

9. The method according to claim 6, wherein:
the individual umbilical cord artery perivascular stem cells is washed twice with the PBS without calcium and magnesium to obtain a cell precipitate, then the cell precipitate is resuspended with the umbilical cord blood-derived platelet-rich plasma, and then gently mixed evenly to obtain the umbilical cord artery perivascular stem cell injection solution; and
the umbilical cord artery perivascular stem cell injection solution is placed at 4° C. for a short-term storage, used within 12 h, and shaken gently before use.

10. A method for preparing the umbilical cord artery perivascular stem cell injection solution for treating the ischemic diseases according to claim 4, comprising:
cultivating an umbilical cord artery tissue to derive perivascular stem cells, then performing an enzymatic digestion on the perivascular stem cells to obtain individual umbilical cord artery perivascular stem cells, followed by washing the individual umbilical cord artery perivascular stem cells twice with PBS without calcium and magnesium to obtain a cell precipitate, and resuspending the cell precipitate with PBS containing 5% (w/v) human serum albumin to obtain a cell suspension solution, and then gently mixing the cell suspension solution evenly to obtain the umbilical cord artery perivascular stem cell injection solution;
wherein the umbilical cord artery perivascular stem cell injection solution is placed at 4° C. for a short-term storage, used within 12 h, and shaken gently before use.
